(19) United States
(12) Patent Application Publication
(10) Pub. No.: US 2026/0334838 A1
(43) Pub. Date: Oct. 29, 2026

(54) SYSTEM AND METHOD FOR ANALYZING MAGNETIC SIGNALS GENERATED BY A HUMAN BODY

(71) Applicant: Robert Bosch GmbH, Stuttgart (DE)

(72) Inventors: Eckhard Wehrse, Stuttgart (DE); Florian Krist, Stuttgart (DE); Florian Dolde, Stuttgart (DE); Frederik Schaal, Reutlingen (DE); Riccardo Cipolletti, Geislingen An Der Steige (DE)

(21) Appl. No.: 19/102,624

(22) PCT Filed: Aug. 18, 2023
(86) PCT No.: PCT/EP2023/072847

§ 371 Date: Feb. 10, 2025

(30) Foreign Application Priority Data
Sep. 9, 2022 (DE) 10 2022 209 442.5

(51) Int. Cl. A61B 5/243; A61B 5/00; G01R 33/26

(52) U.S. Cl. CPC A61B 5/243; A61B 5/704; A61B 5/7246; G01R 33/26; A61B 2562/0223

(57) ABSTRACT

A system for analyzing magnetic signals generated by a human body. The system includes at least one measuring device for detecting magnetic signals and an analysis unit which is configured to store the detected magnetic signals together with an associated measurement time and to derive longitudinal biomagnetic field data from the magnetic signals. The detected magnetic signals and/or the longitudinal biomagnetic field data are compared with one another on different time scales, wherein a vital function of the human body can be evaluated by means of the analysis unit on the basis of the comparison.

### FIELD

The present invention relates to a system and a method for analyzing magnetic signals generated by a human body.

### BACKGROUND INFORMATION

Magnetic fields resulting from the electrophysiological activity of neuronal or muscular tissues can be recorded by various specialized sensor systems and in many cases are used as diagnostic tools. This includes magnetoencephalography, as described for example in Pratt, E. J., Ledbetter, M., Jiménez-Martinez, R., Shapiro, B., Solon, A., Iwata, G. Z., & Alford, J. K. (2021, March), “Kernel Flux: a whole-head 432-magnetometer optically-pumped magnetoencephalography (OP-MEG) system for brain activity imaging during natural human experiences;” In Optical and Quantum Sensing and Precision Metrology (vol. 11700, pp. 162-179). SPIE.), magnetomyography, for example described in Broser, P. J., Middelmann, T., Sometti, D., & Braun, C. (2021), “Optically pumped magnetometers disclose magnetic field components of the muscular action potential;” Journal of Electromyography and Kinesiology, 56, 102490), and magnetocardiography, for example described in Shirai, Y., Hirao, K., Shibuya, T., Okawa, S., Hasegawa, Y., Adachi, Y., . . . & Kawabata, S. (2019), “Magnetocardiography using a magnetoresistive sensor array;” International Heart Journal, 60(1), 50-54.

This involves recording and evaluating time-varying magnetic field data generated by a human body over a period of up to several minutes. In addition to manual evaluation (e.g. by a cardiologist), automatic systems, for example based on neural networks, are also available for the evaluation of such data; see e.g. Tao, R., Zhang, S., Wang, Y., Mi, X., Ma, J., Shen, C., & Zheng, G. (2021), “MCG-Net: End-to-End Fine-Grained Delineation and Diagnostic Classification of Cardiac Events From Magnetocardiographs;” IEEE Journal of Biomedical and Health Informatics, 26(3), 1057-1067.

Measurements of significantly longer duration or periodically recurring measurements (both also referred to as “longitudinal” in the following) currently only take place in isolated cases, due to the technical limitations of the conventional measuring systems.

To measure very small magnetic field strengths, optically pumped quantum sensors or quantum sensors based on NV centers in diamond are particularly suitable as sensors. German Patent Application No. DE 10 2022 204 526.2 describes a magnetometer that uses optically pumped and optically detected magnetic resonances (ODMR). This takes advantage of the fact that under the influence of an external magnetic field, the energy levels of certain spin states of unpaired electrons split, the so-called Zeeman effect. The splitting of the energy levels results in altered transitions during relaxation from excited states, which can then be measured for example by optical excitation and frequency-dependent detection of the resulting fluorescence radiation or by observing optical properties such as the absorption of light. The measured optical parameters can then be used to infer the magnetic field strength.

If such suitable magnetometers are available for recording biomedical fields for long-term investigations, they generate amounts of data that are several orders of magnitude larger than is the case with one-time measurements. On the other hand, only systems for evaluating biomagnetic field data of a few minutes in length are currently in common use. Manual evaluation, on the other hand, must be carried out by trained specialists and usually involves a pairwise comparison of current data with data from earlier points in time. This evaluation is therefore too complex for broad applicability.

If biomagnetic fields are only evaluated at specific points in time, a large part of the available information is not used; for example, rare cardiac arrhythmias can be overlooked.

Furthermore, it may happen that a particular measurement is considered outside its temporal or statistical context. For example, short-term deteriorations in heart muscle function (e.g., within days) cannot be distinguished from longer-term changes that do not currently require treatment.

### SUMMARY

According to the present invention, a device and a method for analyzing magnetic signals generated by a human body are provided. Advantageous example embodiments of the present invention are disclosed herein.

A system according to an example embodiment of the present invention for analyzing magnetic signals generated by a human body includes at least one measuring device for detecting magnetic signals and an analysis unit which is configured to store the detected magnetic signals together with an associated measurement time and to derive longitudinal biomagnetic field data from the magnetic signals. The detected magnetic signals and/or the longitudinal biomagnetic field data are compared with one another on different time scales, wherein a vital function of the human body can be evaluated by means of the analysis unit on the basis of the comparison.

Longitudinal biomagnetic field data are to be understood herein in particular as data sets that describe vital functions of a specific person that are regularly recorded over a longer period of time and that are described by measuring magnetic signals generated by the person's body. For example, the magnetic signals generated by the person's heart, but also signals generated by other muscles or nerves, are suitable for this purpose.

This is a system that develops and tests hypotheses for changes in the electrophysiological activity of certain tissues and organs, or the overall condition of the user, on different time scales.

In a preferred example embodiment of the present invention, for this purpose the magnetic signals are compared on a time scale of a few minutes, wherein in particular a change in the electrical heart axis and/or ST elevations or ST depressions can be detected.

Alternatively or additionally, the magnetic signals can be compared on a time scale of hours to days, wherein in particular signs of right heart strain and/or increases in periods of tachycardie cardiac arrhythmias can be detected.

Alternatively or additionally, the magnetic signals can be compared on a time scale of months to years, wherein in particular long-term changes in resting pulse and heart rate variability can be recognized.

According to an example embodiment of the present invention, the system can preferably process vectorial measured values from a plurality of sensors or gradiometers for the evaluation, can further preferably evaluate the quality of the data (e.g. the signal-to-noise ratio), can further preferably compare the measured values with anonymized data from other users, can further preferably include manual evaluations of previous data in its evaluation, and can communicate with the user. The system can also, further preferably, assess the urgency of a message to the user, relatives and other persons, and can also preferably weigh the need for an escalation of the rescue chain (message on the device, notification of relatives, emergency call).

According to an example embodiment of the present invention, the one measuring device for detecting magnetic signals is preferably designed to record a magnetocardiogram (abbreviated MCG). An MCG is the recording and representation of the magnetic field of the heart, which is created by the electrophysiological activity of the heart muscle cells. This is realized in particular by nitrogen vacancy magnetometers (so-called NV magnetometers) as a measuring device.

In a preferred example embodiment of the present invention, the measuring device comprises a sensor unit for detecting magnetic signals generated by a beating heart, which has a base body with a support surface and an arrangement of at least two NV magnetometer units, wherein the arrangement is embedded in the base body, wherein the base body is configured to accommodate a user sitting or lying on the support surface. Such a device can also be called a magnetocardiograph.

According to an example embodiment of the present invention, a particular advantage of NV sensors is the size of the sensor, specifically the sensor medium or the measuring range. For the application, the active measuring volume should be small compared to the object to be measured (heart), as otherwise the area coverage will result in integration over large parts of the signal and thus the signal may disappear because the integral is zero. The smaller the active measuring volume compared to the heart, the better, in particular more precise and/or accurate, the signal detection. NV sensors have a very small active sensor volume (e.g. a few mm³). This small size also allows the sensors to be used in a geometric arrangement. In particular, very high-resolution arrangements are possible due to the very small active sensor volume.

This also allows for easy integration into textiles or other everyday objects, with numerous options being possible. In one embodiment, the base body is a cushion, a mattress, a lounger, a mat, a bed, a seat (such as a car seat) or a chair; integration is also possible in, for example, a topper, underlay, cover, slatted frame, bed frame, duvet, pillow, side sleeper pillow, etc.

Diamond NV magnetometers are based on the reading of magnetic resonances of specific defect centers in diamond, in particular nitrogen vacancies (NV), which occur as impurities in the carbon lattice of diamond and can also be introduced in a targeted manner. If the NV center is optically excited in the ground state, for example by irradiating a pump laser beam with a suitable wavelength (in this case in the green wavelength range, e.g. at 532 nm for off-resonance excitation), the electrons are lifted from the triplet ground state to the excited triplet state and relax with emission of fluorescent light in the red wavelength range at 650-800 nm (637 nm=zero phonon line).

Since the probability for non-spin-conserving transitions from the spin state with the spin quantum number mₛ=+1 is higher, a continuous excitation pumping ensures that the NV centers are mostly hyperpolarized in the spin state mₛ=0.

Between the mₛ=0 and mₛ=±1 spin states in the ground state there is an energy difference, which in this case is about 2.87 GHz. If microwave radiation is irradiated into the diamond in addition to the optical excitation, there is a collapse in the red fluorescence at this resonance frequency of 2.87 GHz, because the spin-polarized electrons are raised by the microwave field from the mₛ=0 ground state to the mₛ=±1 ground state, and from there are excited by the pump light into the mₛ=±1 excited state. From there, however, mainly non-radiative transitions and weak infrared fluorescence transitions occur via the singlet state, while the fluorescence in the red region disappears.

If an external magnetic field is present, the so-called Zeeman effect causes the splitting of the mₛ=±1 triplet levels, which otherwise have equal energy, into energetically equidistant Zeeman levels. When the fluorescence is plotted against a frequency spectrum of the microwave excitation, two dips are then observed in the fluorescence spectrum whose frequency spacing is proportional to the magnetic field strength of the external magnetic field. The magnetic field sensitivity is mainly defined by the minimum resolvable frequency shift and can reach up to 1 pT/√Hz or better. Since the NV center in single-crystal diamond has four possibilities for situating itself in the crystal lattice, in the presence of a directed magnetic field the NV centers present in the crystal react with varying degrees of strength to the external magnetic field depending on their position in the crystal. In this way, in the ideal case four pairs of fluorescence minima can appear in the spectrum, from whose shape and position relative to each other both the magnetic field strength as a magnitude and the direction of the external magnetic field can be clearly determined.

In order to enable vectorial magnetic field measurements, one example embodiment of the device of the present invention includes an apparatus for generating a substantially homogeneous bias magnetic field in the region of the magnetometer units or their sensor media. This apparatus can also be integrated into the base body. This can be a Helmholtz coil arrangement, wherein at least the sensor medium of the at least two NV magnetometer units is arranged within the Helmholtz coil arrangement. It can also be other apparatuses, such as a simple coil, an elongated coil, permanent magnet solutions such as in a Hallbach array, etc.

Heart signals, which are particularly preferably recorded as magnetic signals within the scope of the present invention, have a magnetic signature with an amplitude of (only) 1 to 2-digit picotesla (pT) at a distance of a few centimeters, whereas, for example, the earth's magnetic field in Central Europe is approximately 50 μT (microtesla), i.e. is stronger by a factor of 10⁶. However, even such small field strengths can be resolved with high accuracy over the long term using the proposed technology. For example, a magnetic shield or a gradiometer circuit can be used for this purpose.

When a gradiometer circuit of at least two NV magnetometer units is used, one magnetometer unit is always at a greater distance from the heart (as a relatively weak magnetic field source) than another magnetometer unit. Due to the gradiometer circuit, i.e. essentially (vectorial) subtraction of what is measured, the magnetic field gradient corresponds approximately to the field emanating from the weak source, while much stronger background fields (which are substantially the same in both magnetometer units) are eliminated. This eliminates the need for magnetic shielding, so that the magnetic field measurement is possible in everyday environments. The present invention is therefore particularly suitable for the unshielded measurement of weak magnetic fields. Technical details of gradiometer solutions that can also be used in the context of the present invention are described in German Patent Application No. DE 10 2022 201 690.4 and are expressly incorporated herein.

In one example embodiment of the present invention, the measuring device is designed to detect a magnetic field strength and field direction by means of each of the at least two NV magnetometer units. Another advantage of NV sensors is the directional or vector information. In contrast to other technologies, this is intrinsic to NV sensors. This means that there is no need to introduce interference through modulation techniques or use less favorable projections, nor is it necessary to use a plurality of separate sensors. This means that the vector and gradiometry information are at exactly the same location (diamond size, i.e., single-digit mm³ and below), and not separated by a few cm to many cm, as with other technologies. With NV magnetometer units, which can determine not only the field strength but also the direction of the magnetic field, improved suppression of a background field and thus better detection of signals that are heavily overlaid by interference signals is possible.

In one example embodiment of the present invention, the measuring device has a signal processing unit to which the at least two NV magnetometer units are connected, wherein the device is configured to use the signal processing unit to determine an effective magnetic field strength and/or an effective magnetic field direction as the difference between the magnetic field strength or field direction detected by the at least two NV magnetometer units. Both a wireless and a wired connection between the sensors and the signal processing unit is provided.

By detecting a magnetic signal, particularly an MCG, and tracking or comparing it over different time scales, a variety of diseases can be detected, such as permanent atrial fibrillation and paroxysmal atrial fibrillation. This can prevent a heart attack and, as a result, a stroke (in particular after an undetected heart attack). Furthermore, the present invention is suitable for the early detection of ST-elevation myocardial infarction, other types of elevation myocardial infarction, pulmonary embolism, AV nodal re-entry tachycardia, ventricular extrasystoles, but also very rare pathogenic diseases such as arrhythmogenic right ventricular tachycardia, which can otherwise only be detected by gene sequencing.

In a further preferred embodiment of a system according to the present invention, additional sensor devices, in particular pressure sensors and/or temperature sensors and/or microphones, can be included in the system. By means of these additional sensor devices, additional biosignals of the human body can preferably be detected, wherein the additional biosignals are included in the evaluation of the vital function by the analysis unit. For example, body weight, body temperature, respiratory rate and/or other values can be recorded and used in the evaluation of vital functions.

Preferably, according to an example embodiment of the present invention, depending on the vital function evaluation, further measures can be initiated, such as sending a report. The system also preferably assesses the urgency of a message to the user, relatives and other persons, and weighs the need for an escalation of the rescue chain (message on the device, notification of relatives, emergency call).

For this purpose, the according to an example embodiment of the present invention, the system preferably has a communication unit designed to send and/or receive data. For this purpose, the communication unit can be designed, for example, to send and/or receive data via a data network, for example the Internet. A connection can be made wired or wirelessly via conventional methods or protocols such as Ethernet, WiFi, Bluetooth, NFC, etc.

According to an example embodiment of the present invention, preferably, data can be received from at least one other system designed according to the present invention and can be included in the evaluation of the vital function by the analysis unit; for example, the recorded signals can be compared with anonymized data from other users and/or manual evaluations of previous data can be included in the evaluation.

For example, a user can be notified when certain vital function parameters deteriorate. In addition, external institutions, relatives, and/or health services can be informed about the development of the vital function by sending corresponding data to them via the communication unit. For example, if there are signs of an acute illness, an emergency service or a doctor can be notified.

The core of the present invention is therefore a system for analyzing and evaluating so-called longitudinal biomagnetic field data, i.e., the observation of the data over a longer period of time and across different time scales.

Such a system can also include other sensors, e.g. pressure sensors, also built into the bed, the mattress, the topper, or under the feet of the bed, in order to observe the long-term development of, e.g., body weight.

According to a second aspect of the present invention, a method for analyzing magnetic signals generated by a human body is provided. According to an example embodiment of the present invention, in the method, magnetic signals, for example MKG signals, from a human body are detected by at least one measuring device, the detected magnetic signals are stored together with an associated measurement time, longitudinal biomagnetic field data are derived from the magnetic signals, and the detected magnetic signals and/or the longitudinal biomagnetic field data are compared with one another on different time scales, wherein an evaluation of a vital function of the human body is carried out based on the comparison. The method can be carried out in particular with a system designed according to the present invention.

Further advantages and embodiments of the present invention can be found in the description and the figures.

The present invention is shown schematically in the figures on the basis of exemplary embodiments and is described below with reference to the figures.

### BRIEF DESCRIPTION OF THE DRAWINGS

FIG. 1 shows a schematic illustration of a system according to an example embodiment of the present invention.

FIG. 2 shows, in a schematic block view, main components of an NV center magnetometer as it can be used in the context of the present invention.

FIG. 3 schematically shows, in a side view, a user on a base body according to an example embodiment of the present invention.

FIG. 4A-4C schematically show an analysis of longitudinal biomagnetic field data on different time scales according to an example embodiment of the present invention.

FIG. 5 schematically shows an analysis of longitudinal biomagnetic field data from a plurality of users on different time scales, according to an example embodiment of the present invention.

### DETAILED DESCRIPTIO OF EXAMPLE EMBODIMENTS

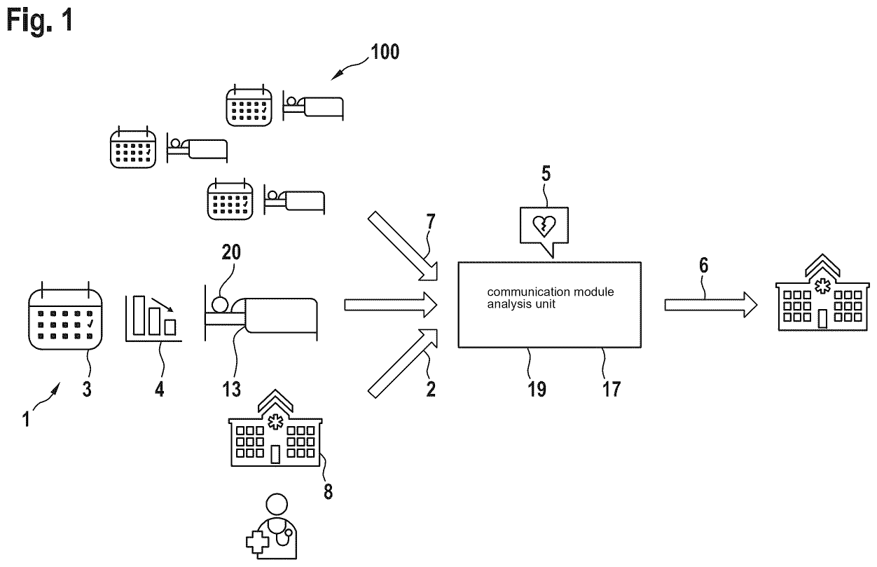

FIG. 1 schematically shows a system 1 according to a possible embodiment of the present invention.

The system 1 for analyzing magnetic signals generated by a human body 20 comprises at least one measuring device 13 for detecting magnetic signals, an analysis unit 19 which is configured to store the detected magnetic signals at regular intervals and continuously together with an associated measurement time (indicated by the calendar) and to derive so-called longitudinal biomagnetic field data 4 from the magnetic signals. The recorded magnetic signals or the biomagnetic field data are compared with each other on different time scales, wherein an evaluation of a vital function of the human body 20 is carried out by the analysis unit 19 based on the comparison.

For the analysis of the longitudinal biomagnetic field data 4 by the system 1, additional data sources are preferably included, for example previous findings 2, which can be obtained from an external data source 8. In addition, anonymized data 7 can be retrieved from other similarly structured or similar systems 100. For data transmission, the analysis unit 19 has a communication module 17. This enables the system 1 to use the biomagnetic field data 4 on different time scales to derive a reasonable suspicion of a disturbance and, if necessary, issue a warning 5 and/or initiate appropriate steps, for example by informing an emergency service 6.

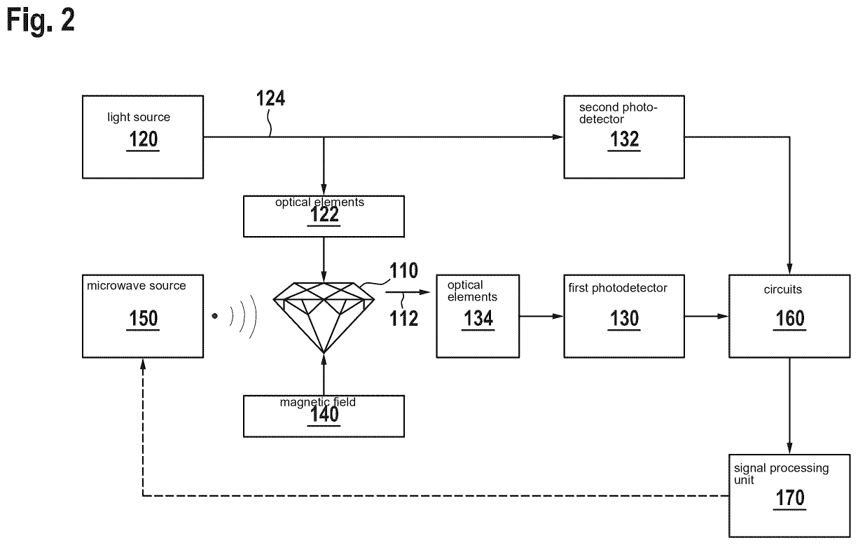

FIG. 2 schematically shows the essential components of an NV center magnetometer, which can advantageously be used as a measuring device in a system according to the present invention. Initially, a diamond 110 with nitrogen vacancies (NV) is provided as the sensor medium. The optical excitation of the NV centers can be achieved by a suitable light source 120 such as a pump laser. For example, a frequency-doubled Nd: YAG laser or semiconductor laser in the green range of about 510-532 nm is suitable here, e.g. at 532 nm for off-resonance excitation. Alternatively, LEDs in suitable wavelength ranges can be used. Depending on the arrangement, the light from the light source 120 can be radiated into the diamond 110 via suitable optical elements 122 such as mirrors, beam splitters, focusing optics such as lenses and, if appropriate, via fiber optic elements. In addition, the excitation light can be emitted continuously or in pulsed form by the laser, so that, for example, time windows can be kept free for interference-free fluorescence light measurement.

Furthermore, the magnetometer may comprise a microwave source 150 capable of generating an electromagnetic field over a bandwidth covering the desired resonance frequency in the sensor medium, i.e. in the region of the NV centers of the diamond 110. A microwave resonator structure can be used to distribute the generated microwaves homogeneously over the volume of the measuring region in the diamond. The resonator structure or the microwave source 150 is preferably tuned to the frequency of the electron spin resonances. To enable vector magnetometry, an additional static bias magnetic field 140 is generated. This makes the measurement intrinsically vectorial. For this purpose, different spatial directions in the crystal structure are used. For example, a Helmholtz coil is suitable for generating such a magnetic field 140, in which a substantially homogeneous magnetic field can be generated in a limited region by means of a pair of coils.

The resulting fluorescent light 112 from the diamond 110 can in turn be guided via suitable optical elements 134 such as optical filters, beam splitters, lenses, and/or fiber optic elements to a first photodetector 130 which is sensitive at least in the range of the fluorescence wavelength. The first photodetector 130 can also be arranged directly on the diamond 110. A second photodetector 132 is arranged such that it can detect at least a portion of the excitation light of the light source 120, which can be coupled out for example by a beam splitter, a filter, or a partially transmissive element. This detector signal 132 of the excitation light can be used as a reference signal, for example to eliminate background signals by modulating the excitation light using a lock-in amplifier and to highlight the resonance signal of interest. Additionally or alternatively, this reference signal can be used to take into account fluctuations in the excitation light. Corresponding circuits 160 such as a preamplifier, a logarithmic amplifier, a lock-in amplifier, signal filters, or others are therefore provided to receive the signals from the first and second photodetectors and to preprocess the signals in a suitable manner for further evaluation. Finally, the preprocessed fluorescence signal can be evaluated by a signal processing unit 170, e.g. with a suitable microcontroller or processor, in order to obtain the desired parameters of the detected magnetic field from the signal, in particular the magnetic field strength and the direction of the magnetic field.

It is understood that such a device can also have other units not shown, such as communication units or interfaces for outputting the measurement results. Such a device can also advantageously be integrated into an ASIC or FPGA.

In order to be usable in an everyday environment, magnetic fields that do not originate from desired weak sources should be eliminated from the measurement as far as possible, in particular the earth's magnetic field in the range of 10-5 tesla (a few microteslas). In contrast, cardiac magnetic fields are in the range of 10-100 times 10-12 tesla (picotesla).

The elimination of the background magnetic fields can be achieved by shielding or by a gradiometer arrangement during magnetic field measurement according to exemplary embodiments. Gradiometers are basically sensor units that are able to record not only the field strength but also the gradient of the field.

For this purpose, at least two individual magnetometer units can be used which are arranged at different spatial locations.

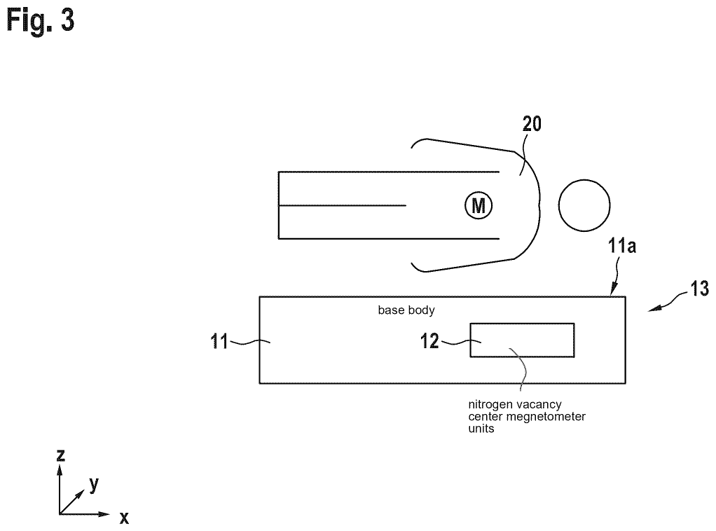

A possible embodiment of the present invention is shown schematically in FIG. 3. A measuring device 13 for detecting magnetic signals is shown, which has a base body 11 with a support surface 11a and at least one arrangement 12 of at least two nitrogen vacancy center (NV) magnetometer units, wherein the at least one arrangement 12 is embedded in the base body 11. The base body is designed to receive a user 20 sitting or lying on the support surface. The measuring device 13 is used to detect magnetic signals generated by a beating heart (M), but can in principle detect all magnetic signals, in particular biosignals, i.e. those emanating from living beings. For illustration purposes, the figure has a coordinate system, wherein the plane of the drawing represents the xz plane and the y axis runs into the plane of the drawing. In FIG. 3, a mattress is shown as an example of a base body 11. It is also possible to design the base body as a blanket, pillow, mattress topper, armchair, seat, car seat, etc.

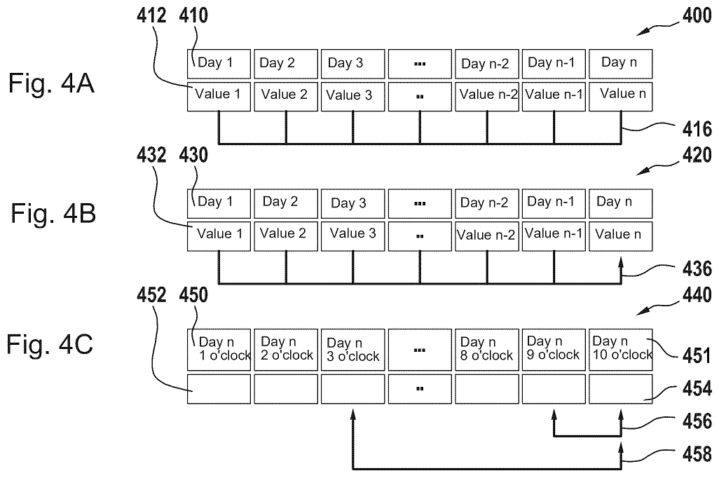

FIGS. 4A-4C show examples of possible evaluations of the recorded magnetic signals. In the example according to FIG. 4A, a table 400 is created in which an associated value 412 is stored for each day 410, wherein the value 412 is calculated using the magnetic signals measured on that day and which in this example represents a nighttime heart rate at rest. The values 412 for each day 410 can then be compared with each other (416), for example to detect a trend or to recognize sudden changes. For example, a health index can be calculated from the curve of the values 412. In addition to the detected magnetic signals, other biosignals can be included that are detected by additional sensors.

In the example according to FIG. 4B, a table 420 is created in which an associated value 432 is stored for each day 430, which is calculated using the magnetic signals measured on that day and which, in this example, also represents a nighttime heart rate at rest. A comparison of a current value 432 with the values of the previous days (426) is carried out and, for example through pattern recognition, deviations can be identified that indicate a pathological change in the state of health.

In the example according to FIG. 4C, a table 440 is created in which an associated value 452 is stored for each hour 450 of a day, which is calculated using the magnetic signals measured in that hour and which, in this example, represents the maximum heart rate during that hour. Various comparisons 456, 458 can now be performed to detect short-term changes in the heart rate. These changes can be assigned to specific events by including additional data sources.

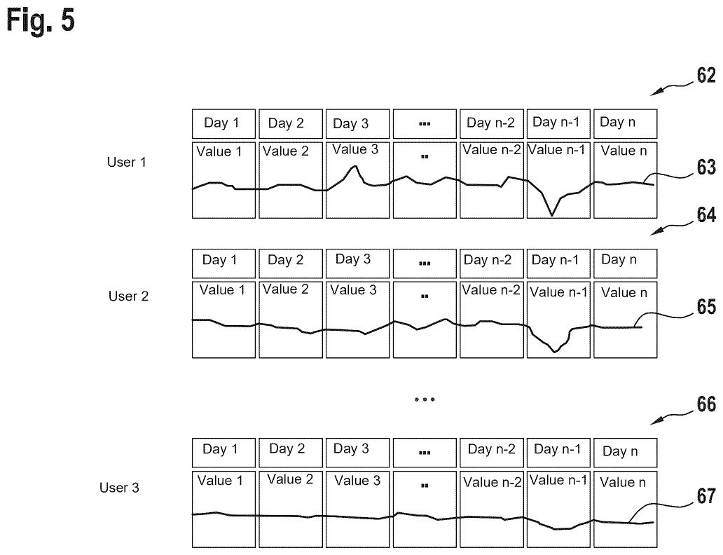

This is shown by way of example in FIG. 5. The curves 62, 64, 66 of a magnetic signal recorded for each of three different persons during the same time period, and a value 63, 65, 67 derived therefrom, which represents for example a sleep quality, are shown here. It is striking that on day 72, a decrease in the recorded value was observed in all subjects. This can be explained, for example, by a particular weather event (e.g. midsummer night) if the people were in the same region. This can be verified by including other sources of information (weather services, Internet, etc.) and taken into account in the further evaluation of the magnetic signals.

1-14. (canceled)

15. A system for analyzing magnetic signals generated by a human body, the system comprising:
at least one measuring device configured to detect magnetic signals; and
an analysis unit configured to store the detected magnetic signals, together with an associated measurement time, at regular time intervals and continuously and/or over a specified period of time, and to derive longitudinal biomagnetic field data from the magnetic signals and to compare the detected magnetic signals and/or the longitudinal biomagnetic field data with one another on different time scales, wherein the analysis unit is configured to evaluate a vital function of the human body based on the comparison.

16. The system according to claim 15, wherein the measuring device is configured to detect magnetic signals generated by a beating heart, and includes a base body, which has a support surface, and an arrangement of at least two nitrogen vacancy center (NV) magnetometer units, wherein the arrangement is embedded in the base body, and wherein the base body is configured to receive a human body sitting or lying on the support surface.

17. The system according to claim 16, wherein the base body includes elastic material between the arrangement and the support surface and is configured as a cushion, or a mattress, or a lounger, or a mat, or a bed, or a seat, or a chair.

18. The system according to claim 16, wherein the measuring device is configured to detect a magnetic field strength and field direction using each of the at least two NV magnetometer units, wherein each of the at least two NV magnetometer units has a diamond crystal or a portion of a diamond crystal having nitrogen vacancy centers as a sensor medium, wherein the device is configured to detect a magnetic field strength and/or field direction by reading out a spin resonance in a sensor medium that is dependent on the magnetic field strength, and wherein the measuring device further includes at least one excitation light source configured to for radiate light into the sensor medium, at least one microwave source configured to generate a resonant field in the sensor medium, and at least one photodetector configured to detect resonance-dependent fluorescent light from the sensor medium.

19. The system as recited in claim 16, wherein the analysis unit or the measuring device includes a signal processing unit to which the at least two NV magnetometer units are connected, wherein the system is configured to determine, using the signal processing unit, an effective magnetic field strength and/or field direction as a difference between magnetic field strengths or field directions detected using the at least two NV magnetometer units as magnetic signals.

20. The system according to claim 15, wherein the analysis unit is configured to compare the magnetic signals and/or the longitudinal biomagnetic field data on a time scale of a few minutes, wherein a change in: (i) an electrical cardiac axis and/or (ii) ST elevations or ST depressions, can be detected.

21. The system according to claim 15, wherein the analysis unit is configured compare the magnetic signals and/or the longitudinal biomagnetic field data on a time scale of hours to days, wherein right heart strain signs and/or increases in periods of tachycardie cardiac arrhythmias are recognizable.

22. The system according to claim 15, wherein the analysis unit is configured to compare the magnetic signals and/or the longitudinal biomagnetic field data on a time scale of months to years, wherein long-term changes in a resting pulse rate and a heart rate variability can be detected.

23. The system according to claim 15, wherein the system further comprises one or more additional sensor devices, wherein the one or more additional sensor devices includes pressure sensors and/or temperature sensors and/or microphones, which detect additional biosignals of the human body, wherein the additional biosignals are included in the evaluation of the vital function by the analysis unit.

24. The system according to claim 15, wherein further measures can be initiated depending on the evaluation of the vital function.

25. The system according to claim 15, wherein the system further comprises a communication unit which is configured to send data and/or receive data.

26. The system according to claim 25, wherein data are received from at least one other system that is configured the same as the system, and the received data are included in the evaluation of the vital function by the analysis unit.

27. The system according to claim 25, wherein the communication unit is configured to transmit an evaluation of the vital function to an external device.

28. A method for analyzing magnetic signals generated by a human body, the method comprising the following steps:
detecting magnetic signals from a human body using at least one measuring device;
storing the detected magnetic signals together with an associated measurement time;
deriving longitudinal biomagnetic field data from the magnetic signals;
comparing the detected magnetic signals and/or the longitudinal biomagnetic field data with one another on different time scales; and
evaluating a vital function of the human body based on the comparison.
